(19) United States
(12) Patent Application Publication
(10) Pub. No.: US 2005/0025737 A1
(43) Pub. Date: Feb. 3, 2005

(54) Compositions containing melon extracts

(76) Inventor: Jean Louis Sebagh, Paris (FR)

Correspondence Address: CISLO & THOMAS, LLP, 233 WILSHIRE BLVD, SUITE 900, SANTA MONICA, CA, 90401-1211, US

(21) Appl. No.: 10/845,466

(22) Filed: May 12, 2004

Related U.S. Application Data
(60) Provisional application No. 60/491,589, filed on Jul. 30, 2003.

(51) Int. Cl. A61K 7/06; A61K 31/665; A61K 35/78

(52) U.S. Cl. 424/74; 424/94.4; 424/94.1; 514/100; 514/18

(57) ABSTRACT

Cosmetic compositions composed of melon fruit extract in combination with magnesium ascorbyl phosphate (MAP). These compositions may be preventative as well as reparative in treating wrinkles and other signs of aging of the skin, for the face and other areas of the body, as well as providing other beneficial effects to the skin. In one exemplary embodiment, the melon extract is obtained from a cucumis melo variety obtained by genetic crossing, such as the 95LS444 line, or one of the hybrids obtained from this line. In another exemplary embodiment, the melon extract may be produced using only the melon pulp without any solvent and may be coated into microgranules with a vegetable fat.

### CROSS-REFERENCE TO RELATED APPLICATIONS

This patent application claims the benefit of U.S. Provisional Patent Application Ser. No. 60/491,589 filed Jul. 30, 2003 for COMPOSITIONS CONTAINING MELON EXTRACT, which application is hereby incorporated by reference.

### BACKGROUND

Skin care formulations containing antioxidants have been developed and used in attempts to prevent free radical damage to the skin. By preventing free radical damage to the skin, these antioxidants may minimize the effects and signs of aging skin. For instance, skin care compositions may contain antioxidant enzymes, such as Superoxide Dismutase (SOD) and Catalase. These antioxidant enzymes are generally considered to be primary antioxidants, yet they are not often used because they are easily damaged and hard to stabilize.

Antioxidant vitamins, such as Vitamin C and Vitamin A, are more commonly used as they are more stable than antioxidant enzymes. While antioxidant vitamins have been known to prevent free radical damage, they are considered secondary antioxidants because they are less effective than primary antioxidants in preventing free radical damage.

Accordingly, there remains a need for skin care compositions containing antioxidant materials that can minimize free radical damage to the skin and thereby improve the appearance of the skin.

### SUMMARY

Embodiments disclosed herein are directed to cosmetic compositions containing natural melon fruit extract and magnesium ascorbyl phosphate (MAP). These compositions may be preventative as well as reparative in treating wrinkles and other signs of aging of the skin, for the face and other areas of the body, as well as providing other beneficial effects to the skin.

Natural melon contains a mixture of ingredients that are beneficial to the skin, including superoxide dismutase (SOD), catalase, vitamins, coenzyme Q10, lipoic acid, glutathion, and mineral salts such as potassium, magnesium, calcium, and selenium. Various antioxidants such as, but not limited to, SOD and catalase minimize free radical damage to the skin, thereby reducing or slowing down the signs of aging. Additionally, MAP works in combination with the melon extract to stimulate collagen production and improve the elasticity of the skin.

In one exemplary embodiment, the melon extract is obtained from a cucumis melo variety obtained by genetic crossing, such as the 95LS444 line, or one of the hybrids obtained from this line (see U.S. Pat. Nos. 5,747,043 and 5,616,323). This melon variety has a typical shelf life of about 14 days, which is approximately three times more than the usual melon variety. In another exemplary embodiment, the extract used in the cosmetic compositions is produced using only the melon pulp without any solvent. In yet another exemplary embodiment, the raw extract is coated into microgranules with vegetable fats. The microgranules may comprise, for example, about 50% extract and about 50% fat, and can be incorporated into cosmetic formulations. These microgranules can provide a strong barrier for antioxidants and bioavailability, thereby protecting and stabilizing the primary antioxidants against moisture, temperature, acidic pH, etc.

The cosmetic compositions may be formulated as creams, gels, pastes, cleansers, mud masks, or other suitable vehicles for application to the skin.

### BRIEF DESCRIPTION OF THE DRAWINGS

FIG. 1 is a graph showing the results of an experiment demonstrating the in-vitro protective effect of microgranules of melon extract on human keratinocytes.

FIG. 2 is a graph showing the results of an experiment demonstrating the ex-vivo protective effect on skin biopsies of microgranules of melon extract on human keratinocytes.

FIG. 3 is a graph showing the results of an experiment demonstrating the protective effect of microgranules of melon extract for the prevention of skin allergies.

### DETAILED DESCRIPTION

The detailed description set forth below in connection with the appended drawings is intended as a description of exemplary embodiments and is not intended to represent the only forms in which the embodiments may be constructed and/or utilized. The description also sets forth the functions and the sequence of steps for constructing and operating the invention in connection with the illustrated embodiments. However, it is to be understood that the same or equivalent functions and sequences may be accomplished by different embodiments that are also intended to be encompassed within the spirit and scope of the invention.

As used herein, “safe and effective amount” means a sufficient amount of a compound, composition or other material described by this phrase to significantly induce a positive modification in the condition being treated, but low enough to avoid undue side effects (e.g., significant skin irritation or sensitization), within the scope of sound judgment of the skilled person. The safe and effective amount of the compound, composition or other material may vary with the particular skin being treated, the age and physical condition of the biological subject being treated, the severity of the condition, the duration of treatment, the nature of concurrent therapy, the specific compound, composition, or other material employed, the particular cosmetically acceptable topical carrier utilized, and the factors within the knowledge and expertise of the skilled person.

According to one exemplary embodiment, the cosmetic compositions contain natural melon fruit extract and magnesium ascorbyl phosphate (MAP). These compositions can be preventative as well as reparative in treating wrinkles and other signs of aging of the skin, for the face and other areas of the body, as well as providing other beneficial effects to the skin. More specifically, the natural melon fruit extract contains a mixture of ingredients which are beneficial to the skin, including, but not limited to, superoxide dismutase (SOD), catalase, is vitamins, coenzyme Q10, lipoic acid, glutathion, and mineral salts such as potassium, magnesium, calcium, and selenium. The antioxidants contained within the melon extract can prevent free radical damage to the skin and thereby reducing or slowing down the signs of aging. MAP works in combination with the melon extract to stimulate collagen production and improve the elasticity of the skin.

In one exemplary embodiment, the melon extract is obtained from a cucumis melo variety obtained by genetic crossing, such as the 95LS444 line, or one of the hybrids obtained from this line (see U.S. Pat. Nos. 5,747,043 and 5,616,323, which are hereby incorporated by reference). This melon variety has a typical shelf life on the order of 14 days, which is approximately three times more than the usual variety. The presence of antioxidants, especially SOD and catalase, explains in part the exceptionally long storage life of these unique melon varieties.

According to another exemplary embodiment, the melon extract can be produced using only the melon pulp without any solvent. Additionally, the raw melon extract is coated into microgranules with vegetable fats. The microgranules may comprise, for example, about 50% extract and about 50% fat, and are easily incorporated in cosmetic formulations. As those skilled in the art will appreciate, the percentages of the extract and fat may be varied according to a desired composition by combining the extract with a vegetable fat. A strong barrier for antioxidants and bioavailability is produced that also protects and stabilizes the antioxidants against moisture, temperature, acidic pH, etc.

The unique melon variety coated into global change microgranules achieves an effective antioxidant activity from approximately 0.1% and has no prooxidant activity.

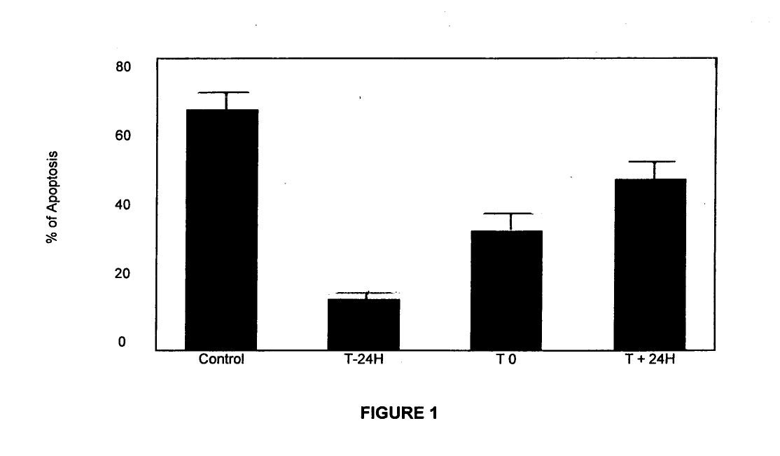

In an experiment demonstrating the in-vitro protective effect on human keratinocytes, the above described microgranules, comprising 50% melon extract and 50% vegetable fat, were introduced close to keratinocytes 24 hours before the UV stress (“T-24H”), to demonstrate preventative effect at the time of ultraviolet (UV) exposure (“T0”) and 24 hours after UV exposure (“T+24H”). Additionally, these results were compared to keratinocytes that were untreated. The keratinocyte apoptosis and the release of some cytokines (TNF, IL-6 and NO) were also monitored. The results, as shown in FIG. 1 and Table I, demonstrate that the microgranules have a preventative effect on keratinocytes by limiting UV induced apoptosis. Also, the microgranules prevent UV exposure-induced inflammation by limiting the production of pro-inflammatory mediators.

TABLE I

| | | UV + | UV + | UV + |
|---|---|---|---|---|
| | | microgranules | microgranules | microgranules |
| | UV | T − 24 h | T = 0 | T + 24 h |
| TNF | 310 ± 75 | 95 ± 12 | 125 ± 6 | 306 ± 17 |
| IL-6 | 255 ± 30 | 100 ± 18 | 108 ± 4 | 212 ± 8 |
| NO | 10 ± 2 | 1.5 ± 2 | 6.5 ± 2 | 9.2 ± 2 |

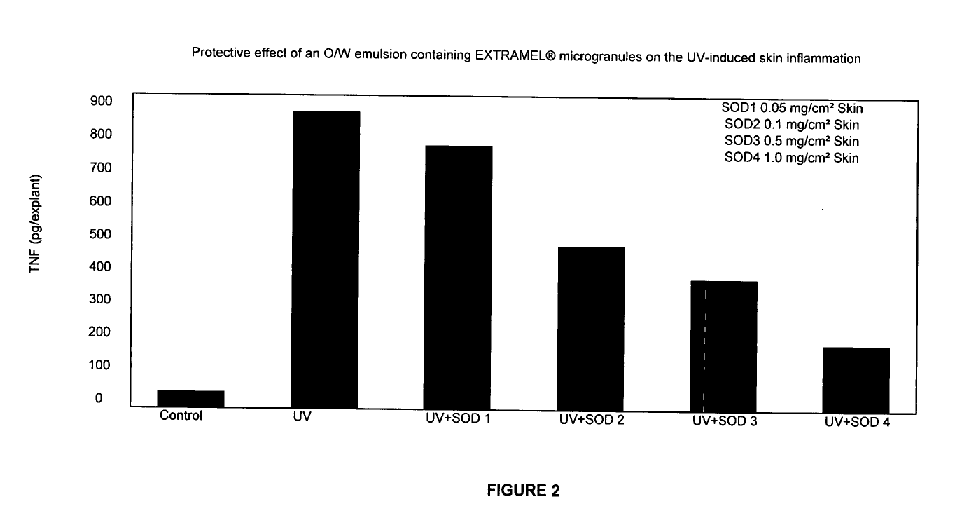

In a further experiment demonstrating the ex-vivo protective effect on skin biopsies, the influence of the microgranules against UV exposure induced inflammation was tested on normal skin biopsies. The microgranules were incorporated at a 1% concentration into an oil in water emulsion. The emulsion was applied on the skin biopsies 24 hours before the UV exposure at the following amounts: 0.05 mg/cm² skin; 0.1 mg/cm²; 0.5 mg/cm²; and 1 mg/cm². The TNF α release was followed as an inflammatory marker. The results, shown in FIG. 2, demonstrate a significant protection of the skin cells against UV by restricting the release of pro-inflammatory markers such as TNFα and thereby limiting the sun induced inflammation.

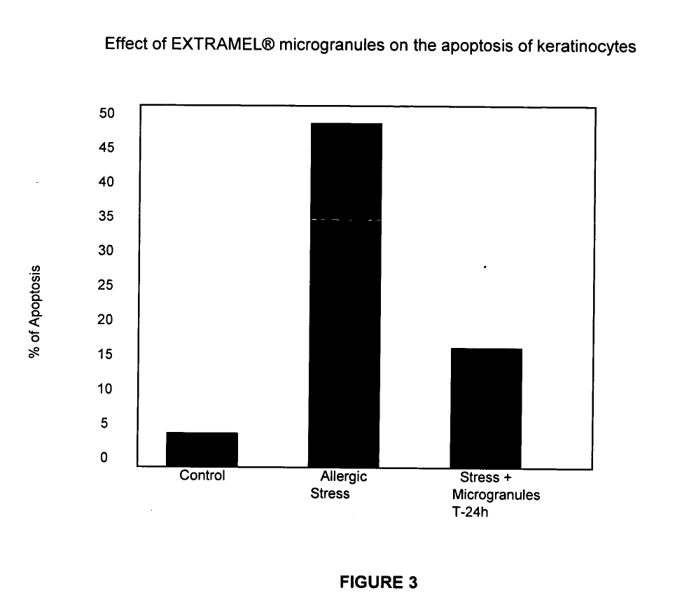

In another experiment, the protective effect of the microgranules for the prevention of skin allergies was demonstrated. The protective effect of the microgranules against allergen substance induced inflammation was carried out using normal human keratinocytes. The microgranules were introduced close to the keratinocytes 24 hours before the allergic stress to demonstrate preventative effect. In order to simulate an allergic stress induced inflammatory recation, the keratinocytes were activated using IL-4 (10 ng/ml) for 48 hours to induce the CD23 (IgE low affinity binding site) and stimulated using IgE immune complexes (IgE/anti-IgE). The cells then were held in the culture medium for 48 hours. The apoptosis of keratinocytes and the release of some inflammatory markers (TNFα, IL-6 and NO) were followed. The results, shown in Table II and FIG. 3, demonstrate a preventative effect on the allergic stress induced inflammation by limiting the production of pro-inflammatory mediators.

TABLE II

| | | | Allergic stress + |
|---|---|---|---|
| | | | microgranules |
| | Control | Allergic stress | T − 24 h |
| TNF | ND | 632 | 152 |
| (pg/ml) | | | |
| IL-6 | ND | 428 | 208 |
| (pg/ml) | | | |
| NO | 1 | 17.3 | 3.6 |
| (uM) | | | |

The results of the above experiments demonstrate that the microgranules of the melon extract have powerful anti-inflammatory properties, would be particularly suitable for anti-pollution, anti-stress treatments, anti-aging products and products for sensitive skin.

The exemplary embodiments of the cosmetic composition may be formulated as creams, gels, pastes, or other suitable vehicles for application to the skin. According to one exemplary embodiment, the cosmetic composition may incorporate approximately 0.5% to 5% by weight of the above-described microgranules, depending on the desired effect. Additionally, the microgranules or compositions containing the microgranules, should be stored at a temperature at or below 40° C. in order to protect the antioxidant enzymes such as, but not limited to, SOD.

The following are examples for preparing an oil in water emulsion and a gel containing the coated microgranules containing a melon extract.

### EXAMPLE 1

### Preparation of an Oil in Water Emulsion

The following ingredients were used in the preparation of an oil in water emulsion:

| Component A: | |
|---|---|
| Macadamia oil | 28% |
| Sorbitan stearate | 3.8% |
| Sorbitan monostearate | 1.2% |
| Component B: | |
| Deionized water | qsp 100% |
| Component C: | |
| Microgranules of melon fruit extract | 1% |
| Macadamia oil | 5% |
| Component D: | |
| Preservative | 0.3% |

The procedure for preparing the emulsion is as follows:
1) Prepare Component A.
2) Heat Components A and B to 70° C.
3) Add Component A to Component B.
4) Stir with an Ultra-Turax and emulsify.
5) Cool down the mixture.
6) At 40° C. while stirring, pour in Components C and D.

The resulting emulsion has a thin texture and a pH of approximately 6.93, and is stable at an ambient temperature of around 40° C.

### EXAMPLE 2

### Preparation of a Gel

The following ingredients were used in the preparation of gel:

| Component A: | |
|---|---|
| Deionized water | qsp 100% |
| Component B: | |
| Thickener | 2% |
| Preservative | 0.3% |
| Component C: | |
| Microgranules of melon fruit extract | 1% |
| Macadamia oil | 10% |

The procedure for preparing the gel is as follows:
1) Introduce the microgranules into the macadamia oil.
2) Add Component B to Component A under mild stirring.
3) Add Component C when the mixture formed in step 2) is homogenous.

The resulting emulsion has a pH of approximately 7.00, and is stable at an ambient temperature of around 40° C.

Cosmetic products in accordance with the present invention may be formulated as gels, lotions, creams, serums, masks, cleansers, etc.

A wide variety of skin care active and inactive ingredients may be advantageously combined with the melon extract and MAP in accordance with the present invention, including, but not limited to, conditioning agents, skin protectants, other antioxidants, UV absorbing agents, sunscreen actives, cleansing agents, viscosity modifying agents, film formers, emollients, surfactants, solubilizing agents, preservatives, fragrance, chelating agents, foaming or antifoaming agents, opacifying agents, stabilizing agents, pH adjustors, absorbents, anti-caking agents, slip modifiers, various solvents, solubilizing agents, denaturants, abrasives, bulking agents, emulsion stabilizing agents, suspending agents, colorants, binders, conditioning agent-emollients, surfactant emulsifying agents, biological products, anti-acne actives, anti-wrinkle and anti-skin atrophy actives, skin barrier repair aids, cosmetic soothing aids, topical anesthetics, artificial tanning agents and accelerators, skin lightening actives, antimicrobial and antifungal actives, sebum stimulators, sebum inhibitors, humectants, and/or combinations thereof.

The following are non-limiting examples of such active and inactive components.

Conditioning Agents:

Conditioning agents may generally be used to improve the appearance and/or feel of the skin upon and after topical application via moisturization, hydration, plasticization, lubrication, and occlusion, or a combination thereof. Non-limiting examples of suitable conditioning agents are described in the CTFA Cosmetic Ingredient Handbook, Second Edition, 1992, which is incorporated by reference herein in its entirety.

According to one exemplary embodiment, the conditioning component may be composed of a water-soluble conditioning agent, an oil soluble conditioning agent, a conditioning emulsion, or any combination or permutation thereof.

Non-limiting examples of oil soluble conditioning agents include, but are not limited to, mineral oil (see The Merck Index, Tenth Edition, Entry 7048, p. 1033 (1983) and International Cosmetic Ingredient Dictionary, Fifth Edition, vol. 1, p.415-417 (1993)), petrolatum (see The Merck Index, Tenth Edition, Entry 7047, p. 1033 (1983); Schindler, Drug. Cosmet. Ind., 89, 36-37, 76, 78-80, 82 (1961), and International Cosmetic Ingredient Dictionary, Fifth Edition, vol. 1, p. 537 (1993)), C7-C40 branched chain hydrocarbons, C1-C30 alcohol esters of C1-C30 carboxylic acids, C1-C30 alcohol esters of C2-C30 dicarboxylic acids, monoglycerides of C1-C30 carboxylic acids, diglycerides of C1-C30 carboxylic acids, triglycerides of C1-C30 carboxylic acids, ethylene glycol monoesters of C1-C30 carboxylic acids, ethylene glycol diesters of C1-C30 carboxylic acids, propylene glycol monoesters of C1-C30 carboxylic acids, propylene glycol diesters of C1-C30 carboxylic acids, C1-C30 carboxylic acid monoesters and polyesters of sugars, polydialkylsiloxanes, polydiarylsiloxanes, polyalkarylsiloxanes, cylcomethicones having 3 to 9 silicon atoms, vegetable oils, hydrogenated vegetable oils, polypropylene glycol C4-C20 alkyl ethers, di C8-C30 alkyl ethers, and mixtures thereof.

Non-limiting examples of straight and branched chain hydrocarbons having from about 7 to about 40 carbon atoms include, but are not limited to, dodecane, isododecane, squalane, cholesterol, hydrogenated polyisobutylene, docosane (i.e. a C.sub.22 hydrocarbon), hexadecane, isohexadecane (a commercially available hydrocarbon sold as Permethyl.RTM. 101A by Presperse, South Plainfield, N.J.). Also useful are the C7-C40 isoparaffins, which are C7-C40 branched hydrocarbons.

Also useful are C1-C30 alcohol esters of C1-C30 carboxylic acids and of C2-C30 dicarboxylic acids, including straight and branched chain materials as well as aromatic derivatives, esters such as monoglycerides of C1-C30 carboxylic acids, diglycerides of C1-C30 carboxylic acids, triglycerides of C1-C30 carboxylic acids, ethylene glycol monoesters of C1-C30 carboxylic acids, ethylene glycol diesters of C1-C30 carboxylic acids, propylene glycol monoesters of C1-C30 carboxylic acids, and propylene glycol diesters of C1-C30 carboxylic acids, including straight chain, branched chain and aryl carboxylic acids, and propoxylated and ethoxylated derivatives of these materials. Examples of these include diisopropyl sebacate, diisopropyl adipate, isopropyl myristate, isopropyl palmitate, myristyl propionate, ethylene glycol distearate, 2-ethylhexyl palmitate, isodecyl neopentanoate, di-2-ethylhexyl maleate, cetyl palmitate, myristyl myristate, stearyl stearate, cetyl stearate, behenyl behenrate, dioctyl maleate, dioctyl sebacate, diisopropyl adipate, cetyl octanoate, diisopropyl dilinoleate, caprilic/capric triglyceride, PEG-6 caprylic/capric triglyceride, PEG-8 caprylic/capric triglyceride, cetyl ricinoleate, cholesterol hydroxystearate, cholesterol isostearate, and mixtures thereof.

Also useful are various C1-C30 monoesters and polyesters of glycerin and related materials, which are derived from glycerin and one or more carboxylic acid moieties. Depending on the constituent acid and glycerin, these esters can be in either liquid or solid form at room temperature. Non-limiting examples of solid esters include glyceryl tribehenate, glyceryl stearate, glyceryl palmitate, glyceryl distearate, and glyceryl dipalmitate.

Also useful are various C1-C30 monoesters and polyesters of sugars and related materials. Depending on the constituent acid and sugar, these esters can be in either liquid or solid form at room temperature. Examples of liquid esters include glucose tetraoleate, the glucose tetraesters of soybean oil fatty acids (unsaturated), the mannose tetraesters of mixed soybean oil fatty acids, the galactose tetraesters of oleic acid, the arabinose tetraesters of linoleic acid, xylose tetralinoleate, galactose pentaoleate, sorbitol tetraoleate, the sorbitol hexaesters of unsaturated soybean oil fatty acids, xylitol pentaoleate, sucrose tetraoleate, sucrose pentaoletate, sucrose hexaoleate, sucrose hepatoleate, sucrose octaoleate, and mixtures thereof. Examples of solid esters include: sorbitol hexaester in which the carboxylic acid ester moieties are palmitoleate and arachidate in a 1:2 molar ratio; the octaester of raffinose in which the carboxylic acid ester moieties are linoleate and behenate in a 1:3 molar ratio; the heptaester of maltose wherein the esterifying carboxylic acid moieties are sunflower seed oil fatty acids and lignocerate in a 3:4 molar ratio; the octaester of sucrose wherein the esterifying carboxylic acid moieties are oleate and behenate in a 2:6 molar ratio; and the octaester of sucrose wherein the esterifying carboxylic acid moieties are laurate, linoleate and behenate in a 1:3:4 molar ratio. An exemplary solid material is sucrose polyester in which the degree of esterification is 7-8, and in which the fatty acid moieties are C18 mono- and/or di-unsaturated and behenic, in a molar ratio of unsaturates:behenic of 1:7 to 3:5. Another exemplary solid sugar polyester is the octaester of sucrose in which there are about 7 behenic fatty acid moieties and about 1 oleic acid moiety in the molecule. Other materials include cottonseed oil or soybean oil fatty acid esters of sucrose.

Nonvolatile silicones such as polydialkylsiloxanes, polydiarylsiloxanes, and polyalkarylsiloxanes are also useful oils. The polyalkylsiloxanes correspond to the general chemical formula R.sub.3 SiO[R₂ SiO]ₓ SiR₃ wherein R is an alkyl group (preferably R is methyl or ethyl, more preferably methyl) and x is an integer up to about 500, chosen to achieve the desired molecular weight. Commercially available polyalkylsiloxanes include the polydimethylsiloxanes, which are also known as dimethicones, non-limiting examples of which include the VICASIL.RTM. series sold by General Electric Company and the DOW CORNING.RTM. 200 series sold by Dow Corning Corporation. Specific examples of polydimethylsiloxanes useful herein include DOW CORNING.RTM. 225 fluid having a viscosity of 10 centistokes and a boiling point greater than 200° C., and DOW CORNING.RTM. 200 fluids having viscosities of 50, 350, and 12,500 centistokes, respectively, and boiling points greater than 200° C. Also useful are materials such as trimethylsiloxysilicate, which is a polymeric material corresponding to the general chemical formula [(CH₂)₃ SiO_{1/2}]ₓ[SiO₂]y, wherein x is an integer from about 1 to about 500 and integer from about 1 to about 500. A commercially available trimethylsiloxysilicate is sold as a mixture with dimethicone as DOW CORNING.RTM. 593 fluid. Also useful herein are dimethiconols, which are hydroxy terminated dimethyl silicones. These materials can be represented by the general chemical formulas R₃SiO[R₂SiO]ₓSiR₂OH and HOR₂SiO[R₂SiO]ₓ SiR₂OH wherein R is an alkyl group (preferably R is methyl or ethyl, more preferably methyl) and x is an integer up to about 500, chosen to achieve the desired molecular weight. Commercially available dimethiconols are typically sold as mixtures with dimethicone or cyclomethicone (e.g. DOW CORNING.RTM. 1401, 1402, and 1403 fluids). Also useful herein are polyalkylaryl siloxanes, with polymethylphenyl siloxanes having viscosities from about 15 to about 65 centistokes at 25° C. being preferred. These materials are available, for example, as SF 1075 METHYLPHENYL FLUID (sold by General Electric Company) and 556 COSMETIC GRADE PHENYL TRIMETHICONE FLUID (sold by Dow Corning Corporation).

Vegetable oils and hydrogenated vegetable oils are also useful herein. Examples of vegetable oils and hydrogenated vegetable oils include safflower oil, castor oil, coconut oil, cottonseed oil, menhaden oil, palm kernel oil, palm oil, peanut oil, soybean oil, rapeseed oil, linseed oil, rice bran oil, pine oil, sesame oil, sunflower seed oil, hydrogenated safflower oil, hydrogenated castor oil, hydrogenated coconut oil, hydrogenated cottonseed oil, hydrogenated menhaden oil, hydrogenated palm kernel oil, hydrogenated palm oil, hydrogenated peanut oil, hydrogenated soybean oil, hydrogenated rapeseed oil, hydrogenated linseed oil, hydrogenated rice bran oil, hydrogenated sesame oil, hydrogenated sunflower seed oil, and mixtures thereof.

Also useful are C4-C20 alkyl ethers of polypropylene glycols, C1-C20 carboxylic acid esters of polypropylene glycols, and di-C8-C30 alkyl ethers. Non-limiting examples of these materials include PPG-14 butyl ether, PPG-15 stearyl ether, dioctyl ether, dodecyl octyl ether, and mixtures thereof.

Other useful oil soluble conditioning agents include CREMEROL (available from Amerchol), ELDEW CL301 (available from Ajinomoto), MODULAN (an acetylated lanolin which is commercially available from Croda), OHLAN (a hydroxylated lanolin which is commercially available from Amerchol), phytantriol, super sterol esters, such as C₁-C₃₀ cholesteroltlanosterol esters, (available from Croda), and mixtures thereof.

Non-limiting examples of conditioning agents useful as water soluble conditioning agents include those selected from the group consisting of polyhydric alcohols, polypropylene glycols, polyethylene glycols, ureas, pyrolidone carboxylic acids, ethoxylated and/or propoxylated C3-C6 diols and triols, alpha-hydroxy C2-C6 carboxylic acids, ethoxylated and/or propoxylated sugars, polyacrylic acid copolymers, sugars having up to about 12 carbons atoms, sugar alcohols having up to about 12 carbon atoms, and mixtures thereof. Specific examples of useful water soluble conditioning agents include materials such as urea; guanidine; glycolic acid and glycolate salts (e.g. ammonium and quaternary alkyl ammonium); lactic acid and lactate salts (e.g. ammonium and quaternary alkyl ammonium); sucrose, fructose, glucose, eruthrose, erythritol, sorbitol, mannitol, glycerol, hexanetriol, propylene glycol, butylene glycol, hexylene glycol, and the like; polyethylene glycols such as PEG-2, PEG-3, PEG-4, PEG-5, PEG-6, PEG-8, PEG-9, PEG-10, PEG-15 PEG-30, PEG-50, polypropylene glycols such as PPG-9, PPG-12, PPG-15, PPG-17, PPG-20, PPG-26, PPG-30, PPG-34; alkoxylated glucose; hyaluronic acid; and mixtures thereof. Also useful are materials such as aloe vera in any of its variety of forms (e.g., aloe vera gel), chitin, starch-grafted sodium polyacrylates such as SANWET (RTM) IM-1000, IM-1500, and IM-2500 (available from Celanese Superabsorbent Materials, Portsmouth, Va.); lactamide monoethanolamine; acetamide monoethanolamine; and mixtures thereof. Also useful are propoxylated glycerols. Other useful water soluble conditioning agents include arginine, arginine asparate (available from Ajinomoto), ARGININE PCA (available from Argidone-UCIB), 1,3 butylene glycol, CHITOLAM NB/101 (available from Lamberti), chitosan salts, Codiavelane (available from Secma), COLLAGEN AMINO ACID (available from Crotein CAA-Croda), creatine, dextran, dextrin, diglycerol, dipropylane glycol, ectoines, erythritol, FUCOGEL (available from Solabia), fructose, glucamine salts, glucose glutamate (commercially available as WICKENOL 545 from Caschem), glucuronic acid salts, glutamic acid salts, glycereth 12, glycereth 20, glycereth 7, glycerin, glyceryl PCA, glycogen, hexylene glycol, honey, hyaluronic acid, hydrogenated honey, hydrogenated starch hydrolysates, hydrolyzed mucopolysaccharides, hydroxy proline, Indinyl CA (available from Laboratoires Serobiologiques), inositol, keratin amino acids (commercially available as CROTEIN HKP from Croda), konjac mannan, Larex A-200 (available from Larex), LYSINE PLA (commercially available as LYSIDONE from UCIB), maltitol, maltose, mannitol, mannose, Mariscan (available from Pentapharm), Melhydrin (available from Laborotories Serobiologiques), methoxy PEG 10, methoxy, methyl gluceth 10 (commercially available as GLUCAM E10 from Amerchol), methyl gluceth 20 (commercially available as GLUCAM E20 from Amerchol), methyl glucose, 3 methyl 1,3 butandiol, N acetyl glucosamine salts, panthenol, PEG 15 butanediol, butanediol, PEG 5 pentaerythitol, pentaerythitol, Pentaglycan (available from Pentapharm), 1,2 pentanediol, phytohyaluron (ute extract), polyglycerol sorbitol, PPG 1 glyceryl ether, proline, propylene glycol, 2 pyrrolidone-5-carboxylic acid and its salts, saccharide isomerate (commercially available as PENTAVITIN from Pentapharm), Seacare (available from Secma), Sericin (available from Pentapharm), serine, silk amino acids (commercially available as CROSLIK LIQUID from Croda), sodium acetylhyaluronate, sodium hyaluronate, sodium polyaspartate (commercially available as AQUADEW SPA-30 from Ajinomoto), sodium polyglutamate (commercially available as AJICOAT SPG from Ajinomoto), sorbeth 20, sorbeth 6, sorbitol, trehalose, triglycerol, trimethyolpropane, tris (hydroxymethyl) amino methane salts, xylitol, xylose, and mixtures thereof.

Sunscreen Actives:

Non-limiting examples of sunscreens which are useful in the compositions include 4-N,N-(2-ethylhexyl)methylaminobenzoic acid ester of 2,4-dihydroxybenzophenone, 4-N,N-(2-ethylhexyl)methylaminobenzoic acid ester with 4-hydroxydibenzoylmethane, 4-N,N-(2-ethylhexyl)-methylaminobenzoic acid ester of 2-hydroxy-4-(2-hydroxyethoxy)benzophenone, 4-N,N-(2-ethylhexyl)-methylaminobenzoic acid ester of 4-(2-hydroxyethoxy)dibenzoylmethane, 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, titanium dioxide, zinc oxide, silica, iron oxide, and mixtures thereof. Other useful sunscreens include aminobenzoic acid (PABA), benzylidene camphor, butyl methoxy dibenzoyl methane, diethanolamine p-methoxycinnamate, dioxybenzone, ethyl dihydroxypropyl (PABA), glyceryl aminobenzoate, homomenthyl salicylate, isopropyl dibenzoyl methane, lawsone and dihydroxyacetone, menthyl anthranilate, methyl anthranilate, methyl benzylidene camphor, octocrylene, octyl dimethyl (PABA), octyl methoxycinnamate, oxybenzone, 2-phenylbenzimidazole-5-sulfonic acid, red petrolatum, sulisobenzone, titanium dioxide, triethanolamine salicylate, zinc oxide, and mixtures thereof. Exact amounts of sunscreens which can be employed will vary depending upon the sunscreen chosen and the desired Sun Protection Factor (SPF) to be achieved.

Anti-Acne Actives:

Non-limiting examples of useful anti-acne actives include the keratolytics such as salicylic acid (o-hydroxybenzoic acid), derivatives of salicylic acid such as 5-octanoyl salicylic acid and 4 methoxysalicylic acid, and resorcinol; retinoids such as retinoic acid and its derivatives (e.g., cis and trans); sulfur-containing D and L amino acids and their derivatives and salts, particularly their N-acetyl derivatives, a preferred example of which is N-acetyl-L-cysteine; lipoic acid; antibiotics and antimicrobials such as benzoyl peroxide, octopirox, tetracycline, 2,4,4′-trichloro-2′-hydroxy diphenyl ether, 3,4,4′-trichlorobanilide, azelaic acid and its derivatives, phenoxyethanol, phenoxypropanol, phenoxyisopropanol, ethyl acetate, clindamycin and meclocycline; sebostats such as flavonoids and bioflavonoids; bile salts such as scymnol sulfate and its derivatives, deoxycholate, and cholate; abietic acid; adapalene; allantoin; aloe extracts; arbietic acid and its salts; aryl-2,4 dioxo oxazolidine derivatives; ASEBIOL (available from Laboratories Serobiologiques, located in Somerville, N.J.); azaleic acid; barberry extracts; bearberry extracts; belamcanda chinensis; benzoquinolinones; benzoyl peroxide; berberine; BIODERMINE (available from Sederma, located in Brooklyn, N.Y.); bioflavinoids; bisabolol; S-carboxymethyl cysteine; carrot extracts; cassin oil; clove extracts; citral; citronellal; climazole; Completech MBAC-OS (available from Lipo); CREMOGEN M82 (available from Dragoco, located in Totowa, N.J.); cucumber extracts; dehydroacetic acid and its salts; dehydroeplandersterone salicylate; dichlorophenyl imidazoldioxolan which is commercially available as COMPLETECH MBAC-OS (from Lipo, located in Paterson, N.J.); DL valine and its esters; DMDM hydantoin; Epicutin TT (available from CLR); erythromycin; escinol; ethyl hexyl monoglyceryl ether; ethyl 2-hydroxy undecanoate; farnesol; farnesol acetate; geranoil; glabridin; gluconic acid; gluconolactone; glyceryl monocaprate; glycolic acid; grapefruit seed extract; gugu lipid; Hederagenin (available from Maruzen); hesperitin; hinokitol; hops extract; hydrogenated rosin; 10 hydroxy decanoic acid; ichtyhol; interleukin 1 alpha antagonists; iodo-2-propynyl butyl carbamate; Kapilarine (available from Greentech); ketoconazole; lactic acid; lemon grass oil; Lichochalcone LR15 (available from Maruzen); linoleic acid; LIPACIDE C8CO (available from Seppic, located in Paris, France); lovastatin; 4 methoxysalicylic acid; metronidazole; minocycline; mukurossi; neem seed oil; vitamin B3 compounds (such as niacinamide and nicotinic acid); nisin; 5-octanoly salicylic acid; octopirox; panthenol; 1-pentadecanol; peonia extract; peppermint extract; phelladendron extract; 2-phenyl-benzothiophene derivatives; phloretin; PHLOROGINE (available from Secma); phosphatidyl choline; proteolytic enzymes; quercetin; red sandalwood extract; resorcinol; rosemary extract; rutin; sage extract; salicin; salicylic acid; skull cap extract; siber hegner extract; siberian saxifrage extract; silicol; sodium lauryl sulfate; sodium sulfoacetamide; Sophora Extract (available from Maruzen); sorbic acid; sulfur; sunder vati extract; tea tree oil; tetracyline; tetra hydroabietic acid; thyme extract; tioxolone; tocopherol; trehalose 6-undecylenoate; 3 tridecene-2-ol; triclosan; tropolone; UNITRIENOL T27 (available from Unichem, located in Gouda, Netherlands); vitamin D.sub.3 and its analogs; white thyme oil; willow bark extract; wogonin; Ylang Ylang; zinc glycerolate; zinc linoleate; zinc oxide; zinc pyrithione; zinc sulfate and mixtures thereof.

Antimicrobial and Antifungal Actives:

Non-limiting examples of antimicrobial and antifungal actives include beta-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentarnidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, octopirox, parachlorometa xylenol, nystatin, tolnaftate, zinc pyrithione; clotrimazole; alantolactone; isoalantolactone; alkanet extract (alaninin); anise; arnica extract (helenalin acetate and 11, 13 dihydrohelenalin); Aspidium extract (phloro, lucinol containing extract); barberry extract (berberine chloride); bay sweet extract; bayberry bark extract (myricitrin); benzalkonium chloride; benzethonium chloride; benzoic acid and its salts; benzoin; benzyl alcohol; blessed thistle; bletilla tuber; bloodroot; bois de rose oil; burdock; butyl paraben; cade oil; CAE (available from Ajinomoto, located in Teaneck, N.J.); cajeput oil; Cangzhu; capsicum frutescens extract; caraway oil; cascarilla bark (sold under the tradename ESSENTIAL OIL); cedarleaf oil; chamomille; chaparral; chlorhexidine gluconate; chlorophenesin; chlorxylenol; cinnamon oil; citronella oil; clove oil; Crinipan AD (available from Climbazole); 2,3-dihydro-farnesol; dehydroacetic acid and its salts; dill seed oil; DOWICIL 200 (available from Dow Chemical, located in Midland, Mich.); echinacea; elenolic acid; epimedium; ethyl paraben; Fo-Ti; galbanum; garden bumet; GERMALL 115 and GERMALL II (available from ISP-Sutton Labs, located in Wayne, N.J.); German chamomile oil; giant knotweed; GLYDANT (available from Lonza, located in Fairlawn, N.J.); GLYDANT PLUS (available from Lonza); grapefruit seed oil; 1,6 hexanediol; hexamidine diisethionate; hinokitiol; honey; honeysuckle flower; hops; immortelle; iodopropynl butyl carbamide (available from Lonza); isobutyl paraben; isopropyl paraben; JM ACTICARE (available from Microbial Systems International, located in Nottingham, NG); juniper berries; KATHON CG (available from Rohm and Haas, located in Philadelphia, Pa.); kojic acid; labdanum; lavender; lemon balm oil; lemon grass; methyl paraben; mint; mume; mustard; myrrh; neem seed oil; ortho phenyl phenol; olive leaf extract (available from Bio Botanica); parsley; patchouly oil; peony root; 1,2 pentandiol; PHENONIP (available from Nipa Labs, located in Wilmington, Del.); phenoxyethanol; phytosphingosine; pine needle oil; PLANSERVATIVE (available from Campo Research); propyl paraben; purslane; quillaira; rhubarb; rose geranium oil; rosemary; sage; salicylic acid; sassafras; savory; sichuan lovage; sodium meta bisulfite; sodium sulfite; SOPHOLIANCE (available from Soliance, located in Compiegne, France); sorbic acid and its salts; sphingosine; stevia; storax; sucrose esters; tarmic acid; tea; tea tree oil (cajeput oil); thyme; triclosan; triclocarban; tropolone; turpentine; umbelliferone (antifungal); yucca; and mixtures thereof.

Anti-Wrinkle, Anti-Skin Atrophy and Skin Repair Actives:

Other anti-wrinkle actives may also be combined with exemplary cosmetic compositions disclosed herein. Non-limiting examples of anti-wrinkle and anti-skin atrophy actives include retinoic acid and its derivatives (e.g., cis and trans); retinal; retinol; retinyl esters such as retinyl acetate, retinyl palmitate, and retinyl propionate; vitamin B₃ compounds (such as niacinamide and nicotinic acid), salicylic acid and derivatives thereof (such as 5-octanoyl salicylic acid, heptyloxy 4 salicylic acid, and 4-methoxy salicylic acid); sulfur-containing D and L amino acids and their derivatives and salts, particularly the N-acetyl derivatives, a preferred example of which is N-acetyl-L-cysteine; thiols, e.g. ethane thiol; hydroxy acids, phytic acid, lipoic acid; lysophosphatidic acid; skin peel agents (e.g., phenol and the like); Actein 27-Deoxyactein Cimicifugoside (available from Cirnigoside); adapalene; ademethionine; adenosine; aletris extract; alkyl glutathione esters; alkoxyalkoxy alkoxyn benzoic and derivatives; aloe derived lectins; amino propane phosphoric acid; 3-aminopropyl dihydrogen phosphate; Amadorine (available from Barnet Products); anise extracts; AOSINE (available from Secma); arginine amino benzoate; ASC III (available from E. Merck, located in Darmstadt, Germany); ascorbic acid; ascorbyl palmitate; asiatic acid; asiaticosides; ARLAMOL GEO.TM. (available from ICI, located in Wilmington, Del.); azaleic acid; benzoic acid derivatives; bertholletia extracts; betulinic acid; BIOCHANIN A AND BIOPEPTIDE CL (available from Sederma, located in Brooklyn, N.Y.); BIOPEPTIDE EL (available from Sederma); biotin; blackberry bark extract; blackberry lily extracts; black cohosh extract; blue cohesh extract; butanoyl betulinic acid; carboxymethyl 1,3 beta glucan; catecholamnines; chalcones; citric acid esters; chaste tree extract; clover extracts; coumestrol; CPC Peptide (available from Barnet Products); daidzein; dang gui extract; darutoside; debromo laurinterol; 1-decanoyl-glycero-phosphonic acid; dehydrocholesterol; dehydrodicreosol; dehydrodieugenol; dehydroepiandersterone; DERMOLECTINE (available from Sederma); dehydroascorbic acid; dehydroepiandersterone sulfate; dianethole; dihydroxy benzoic acid; 2,4 dihydroxybenzoic acid; diglycol guanidine succinate; diosgenin; disodium ascorbyl phosphate; dodecanedioic acid; Ederline (available from Seporga); Enderline (available from Laboratories Seporga); equol; eriodictyol; estrogen and its derivatives; ETF (available from Laboratories Seporga); ethocyn; ELESERYL SH (available from Laboratories Serobiologiques, located in Somerville, N.J.); ENDONUCLEINE (available from Laboratories Serobiologiques); ergosterol; eythrobic acid; fennel extract; fenugreek seed extract; FIBRASTIL (available from Sederma); FIBROSTIMULINES S and P (available from Sederma); FIRMOGEN LS 8445 (available from Laboratories Serobiologiques); formononetin; forsythia fruit extract; gallic acid esters; gamma amino butyric acid; GATULINE RC (available from Gattlefosse, located in Priest, France); genistein; genisteine; genistic acid; gentisyl alcohol; gingko bilboa extracts; ginseng extracts; ginsenoside (RO, R.sub.6-1, R.sub.6-2, R.sub.6-3, R.sub.C, R.sub.D, R.sub.E, R.sub.F, R_{F-2}, R_{G-1}, R_{G-2}); gluco pyranosyl-L-ascorbate; glutathione and its esters; glycitein; hesperitin; hexahydro curcumin; HMG-coenzyme A reductase inhibitors; hops extracts; 11 hydroxy undecanoic acid; 10 hydroxy decanoic acid; 25-hydroxycholesterol; 7-hydroxylated sterols; hydroxyethyl isostearyloxy isopropanolamine; hydroxy-tetra methyl piperidinyloxy; hypotaurine; ibukijakou extract; isoflavone SG 10 (available from Barnet Products); kinetin; kohki extract; L-2-OXO-thiazolidine-4-carboxylic acid esters; lactate dehydrogenase inhibitors; 1-lauryl, -lyso-phosphatidyl choline; lectins; lichochalcone LF15 (available from Maruzen); licorice extracts; lignan; lumisterol; lupenes; luteolin; lysophosphitidic acid; margin; melatonin; melibiose; metalloproteinase inhibitors; methoprene; methoprenic acid; mevalonic acid; MPC COMPLEX (available from CLR); N methyl serine; N methyl taurine; N, N. sup. 1-bis (lactyl) cysteamine; naringenin; neotigogenin; o-desmethylangoiensin; oat beta glucan; oleanolic acid; pantethine; phenylalanine; photoanethone; piperdine; placental extracts; pratensein; pregnenolone; pregnenolone acetate; pregnenolone succinate; premarin; quillaic acid; raloxifene; REPAIR FACTOR 1 and REPAIR FACTOR FCP (both available from Sederna); retinoates (esters of C.sub.2 -C.sub.20 alcohols); retinyl glucuronate; retinyl linoleate; S-carboxymethyl cysteine; SEANAMINE FP (available from Laboratories Serobiologiques); sodium ascorbyl phosphate; soya extracts; spleen extracts; tachysterol; taurine; tazarotene; tempol; thymulen; thymus extracts; thyroid hormones; tigogenin; tocopheryl retinoate; toxifolin; traumatic acid; tricholine citrate; trifoside; uracil derivatives; ursolic acid; vitamin D₃ and its analogs; vitamin K; vitex extract; yam extract; yamogenin; zeatin; and mixtures thereof.

Skin Barrier Repair Actives:

Skin barrier repair actives are those skin care actives that can help repair and replenish the natural moisture barrier function of the epidermis. Non-limiting examples of skin barrier repair actives include Alpha Lipid (available from Lucas Meyer); ascorbic acid; biotin; biotin esters; brassicasterol; caffeine; campesterol; canola derived sterols; Cennamides (available from Ennagram); Ceramax (available from Alban Muller); CERAMAX (available from Quest, located in Ashford, England); CERAMIDE 2 and CERAMIDE HO3.TM. (both available from Sederma); CERAMIDE II (available from Quest); CERAMIDE III and IIIB (both available from Cosmoferm, located in Deft, Netherlands); CERAMIDE LS 3773 (available from Laboratories Serobiologiques); CERAMINOL (available from Inocosm); Cerasol and Cephalip (both available from Pentapharm); cholesterol; cholesterol hydroxystearate; cholesterol isostearate; 7 dehydrocholesterol; DERMATEIN BRC and DERMATEIN GSL (both available from Hormel); ELDEW CL 301 AND ELDEW PS 203 (both available from Ajinomoto); Fitobroside (available from Pentapharm); galactocerebrosides; Generol 122 (available from Henkel); glyceryl serine amide; hydroxyethyl isostearyl isopropanolamine; lactic acid; Lactomide (available from Pentapharm); lanolin; lanolin alcohols; lanosterol; lauric acid N laurylglucamide; lipoic acid; N-acetyl cysteine; N-acetyl-L-serine; N-methyl-L-Serine; Net Sterol-ISO (available from Barnet Products); vitamin B3 compounds (such as niacinamide and nicotinic acid); palmitic acid; panthenol; panthetine; phosphodiesterase inhibitors; PHYTO/CER (available from Intergen); phytoglycolipid millet extract (available from Barnet Products Distributer, located in Englewood, N.J.); PHYTOSPHINGOSINE (available from Gist Brocades, located in King of Prussia, Pa.); PSENDOFILAGGRIN (available from Brooks Industries, located in South Plainfield, N.J.); QUESTAMIDE H (available from Quest); serine; sigmasterol; sitosterol; soybean derived sterols; sphingosine; sphingomylinase; S-lactoyl glutathione; stearic acid; Structurine (available from Silah); SUPER STEROL ESTERS (available from Croda); thioctic acid; THSC CERAMIDE OIL (available from Campo Research); trimethyl glycine; tocopheryl nicotinate; vitamin D.sub.3 ; Y2 (available from Ocean Pharmaceutical); and mixtures thereof.

Cosmetic Soothing Actives:

Cosmetic soothing actives can be effective in preventing or treating inflammation of the skin. Non-limiting examples of cosmetic soothing agents include the following categories: propionic acid derivatives; acetic acid derivatives; fenamic acid derivatives; biphenylcarboxylic acid derivatives; and oxicams. Non-limiting examples of useful cosmetic soothing actives include acetyl salicylic acid, ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen, bucloxic acid, absinthium, acacia, aescin, alder buckthorn extract, allantoin, aloe, APT (available from Centerchem), arnica, astragalus, astragalus root extract, azulene, Baicalin SR 15 (available from Barnet Products Dist.), baikal skullcap, baizhu, balsam canada, bee pollen, BIOPHYTEX (available from Laboratories Serobiologiques), bisabolol, black cohosh, black cohosh extract blue cohosh, blue cohosh extract, boneset, borage, borage oil, bradykinin antagonists, bromelain, calendula, calendula extract, Canadian Willowbark Extract (available from Fytokem), candelilla wax, Cangzhu, canola phytosterols, capsicum, carboxypeptidase, celery seed, celery stem extract, CENTAURIUM (available from Sederma), centaury extract, chamazulene, chamomile, chamomile extract, chaparral, chaste tree, chaste tree extract, chickweed, chicory root, chicory root extract, chirata, chishao, collodial oatmeal, comfrey, comfrey extract, CROMOIST CM GLUCAN (available from Croda), darutoside, dehurian angelica, devil's claw, divalent metals (such as, magnesium, strontium, and manganese), doggrass, dogwood, Eashave (available from Pentapharm), eleuthero, ELHIBIN (available from Pentapharm), ENTELINE 2 (available from Secma), ephedra, epimedium, esculoside; ethacrynic acid, evening primrose, eyebright, Extract LE-100 (available from Sino Lion), Fangfeng, feverfew, ficin, forsythia fruit, Fytosterol 85 (available from Fytokem), ganoderma, gaoben, Gatuline A (available from Gattefosse), gentian, germanium extract, gingko bilboa extract, ginkgo, ginseng extract, goldenseal, gorgonian extract, gotu kola, grape fruit extract, guaiac wood oil, guggal extract, helenalin esters, henna, honeysuckle flower, horehound extract, horsechestnut, horsetail, huzhang, hypericum, ichthyol, immortelle, ipecac, job's tears, jujube, kola extract, LANACHRYS 28 (available from Lana Tech), lemon oil, lianqiao, licorice root, ligusticum, ligustrum, lovage root, luffa, mace, magnolia flower, manjistha extract, margaspidin, matricin, melatonin, MICROAT IRC (available from Nurture), mints, mistletoe, Modulene (available from Seporga), mono or diglucosides of glabridin, mono or diglucosides of gentisin, MTA (5′-deoxy-5′-methythioadenosine), mung bean extract, musk, N-methyl arginine, oat beta glucan, oat extract, orange, panthenol, papain, phenoxyacetic acid, peony bark, peony root, Phytoplenolin (available from Bio Botanica), phytosphingosine, Preregen (available from Pentapharm), purslane, QUENCH T (available from Centerchem), quillaia, red sage, rehmannia, rhubarb, rosemary, rosmarinic acid, royal jelly, rue, rutin, sandlewood, sanqi, sarsaparilla, saw palmetto, SENSILINE (available from Silab), SIEGESBECKIA (available from Sederma), stearyl glycyrrhetinate, Stimutex (available from Pentapharm), storax, strontium nitrate, sweet birch oil, sweet woodruff, tagetes, tea extract, thyme extract, tienchi ginseng, tocopherol, tocopheryl acetate, triclosan, turmeric, urimei, ursolic acid, white pine bark, witch hazel xinyi, yarrow, yeast extract, yucca, and mixtures thereof.

Artificial Tanning Actives and Accelerators:

Non-limiting examples of artificial tanning agents and accelerators include dihydroxyacetaone; tyrosine; tyrosine esters such as ethyl tyrosinate and glucose tyrosinate; acetyl tyrosine; phospho-DOPA; brazilin; caffeine; coffee extracts; dihydroxyacetone; DNA fragments; isobutyl methyl xanthine; methyl xanthine; Phototan (available from Laboratoires Serobiologiques); prostaglandins; tea extracts; theophylline; tyrosine; UNIPERTAN P2002 and UNIPERTAN P27 (both available from Unichem); and mixtures thereof.

Skin Lightening Actives:

Skin lightening actives can decrease the amount of melanin in the skin or provide an such an effect by other mechanisms. Non-limiting examples of skin lightening actives useful herein include adapalene, aloe extract, alpha-glycaryl-L-ascorbic acid, aminotyroxine, ammonium lactate, anethole derivatives, apple extract, arbutin, areca catechu L. extract, ascorbic acid, ascorbyl palmitate, azelaic acid, bamboo extract, bearberry extract, bletilla tuber, bupleurum falcatum extract, burnet extract, Burnet Power (available from Barnet Products), butyl hydroxy anisole, butyl hydroxy toluene, butyl resoreinol, Chuanxiong, cola decaballo extract, Dang-Gui, deoxyarbutin, 1,3 diphenyl propane derivatives, 2,5 dihydroxybenzoic acid and its derivatives, 2-(4-acetoxyphenyl)-1,3 dithane, 2-(4-hydroxyphenyl)-1,3 dithane, ellagic acid, escinol, estragole derivatives, esculoside, esculetin, FADEOUT (available from Pentapharm), Fangfeng, fennel extract, gallic acid and its derivatives, ganodenna extract, gaoben, GATULINE WHITENING (available from Gattlefosse), genistic acid and its derivatives, gentisyl alcohol, glabridin and its derivatives, gluco pyranosyl-1-ascorbate, gluconic acid, glucosamine, glycolic acid, glycyrrhizinic acid, green tea extract, 4-Hydroxy-5-methyl-3[2H]-furanone, hydroquinine, 4 hydroxyanisole and its derivatives, 4-hydroxy benzoic acid derivatives, hydroxycaprylic acid, hyptis extract, inositol ascorbate, kojic acid, kojic dipalnitate, lactic acid, lemon extract, licorice extract, Licorice P-TH (available from Barnet Products), linoleic acid, Melfade (available from Pentapharm), MELAWHITE (available from Pentapharm), Melanostatine DM (available from Laboratories Seporga), morus alba extract, mulberry root extract, niacinamide, 5-octanoyl salicylic acid, parsley extract, phellinus linteus extract, pinon blanco extract, pinon negro extract, piri-piri extract, pyrogallol derivatives, retinoic acid, retinol, retinyl esters (acetate, propionate, palmitate, linoleate), 2,4 resorcinol derivatives, 3,5 resorcinol derivatives, rose fruit extract, rucinol, salicylic acid, Song-Yi extract, Sophora Powder (available from Barnet Products), 4-thioresorein, 3,4,5 trihydroxybenzyl derivatives, tranexamic acid, tyrostat (Rumex Extract available from Fytokem), Tyroslat 10,11 (available from Fytokem), vanilla derivatives, vitamin D.sub.3 and its analogs, and mixtures thereof.

Sebum Stimulators:

Sebum stimulators can increase the production of sebum by the sebaceous glands. These skin care actives are especially useful for post menopausal women who are sebum deficient. Non-limiting examples of sebum stimulating actives include bryonolic acid, completech MBAC-DS, dehydroetiandrosterone (also known as DHEA), orizanol and mixtures thereof.

Sebum Inhibitors:

Sebum inhibitors can decrease the production of sebum by the sebaceous glands. Non-limiting examples of sebum inhibiting actives include aluminium hydroxy chloride, ASEBIOL (available from Laboratories Serobiologiques), BIODERMINE (available from Sederma), climbazole, COMPLETECH MBAC-OS (available from Lipo), corticosteroids, cucumber extracts, dehydroacetic acid and its salts, dichlorophenyl imidazoldioxolan (available from Elubiol), gugulipiu, ketoconazole, Lichochalcone LR 15 (available from Maruzen), niacinamide, phloretin, PHLOROGINE (available from Secma), Phycosaccharide Anti-Acne (available from Codif), S-carboxylmethyl cysteine, sepicontrol AS, spironolactone, tioxolone, tocopherol, tranexamic acid, UNITRIENOL T27 (available from Unichem), zincidone (UC1B), and mixtures thereof.

Protease Inhibitors:

Non-limiting examples of protease inhibitors which are useful in the exemplary cosmetic compositions are selected from the group consisting of A E Complex (available from Barnet Products); ALE (available from Seporga); allicin; alpha lupaline; Aosaine (available from Secma); Aprotinin (available from Pentapharm); areca catechu (Betel Nut) extract; areca catechu extracts; Blue Algae Extract (available from Collaborative Labs); Centaurium (available from Sederma); cholesterol sulfate; CMST (available from Bioetica); Dermoprotectine (available from Sederma); Disacoside HF 60 (available from Barnet Products); Elhibin (available from Pentapharm); Fluid Out Colloid (available from Vegetech); Hypotaurine (available from Sogo Pharmaceutical); In Cyte Heathes (available from Collaborative Labs); Micromerol (available from Collaborative Labs); Pefabloc SP (available from Pentapharm); Sepicontrol AS (available from Seppic); Siegesbeckia (available from Sederma); Sophorine (available from Barnet Products); Thiotaine (available from Barnet Products); uncaria gambis roxburgh extract; zinc and mixtures thereof.

Skin Tightening Agents:

The cosmetic compositions may also include skin-tightening agents as active ingredients. Non-limiting examples of skin tightening agents which are useful in the compositions of the present invention are those selected from the group consisting of Biocare SA (available from Amerchol); egg albumen; Flexan 130 (available from National Starch); Gatuline Lifting (available from Gattefosse); Pentacare HP (available from Pentapharm); Vegeseryl (available from Laboratories Serobioloques) and mixtures thereof.

Viscosity Modifiers:

Suitable thickeners and viscosity modifiers include water-soluble polyacrylic and hydrophobically modified polyacrylic resins such as Carbopol and Pemulen, starches such as corn starch, potato starch, tapioca, gums such as guar gum, gum arabic, cellulose ethers such as hydroxypropyl cellulose, hydroxyethyl cellulose, carboxymethyl cellulose, and the like.

Emulsifiers:

A wide variety of emulsifiers are useful herein and include, but not limited to, sorbitan esters, glyceryl esters, polyglyceryl esters, methyl glucose esters, sucrose esters, ethoxylated fatty alcohols, hydrogenated castor oil ethoxylates, sorbitan ester ethoxylates, polymeric emulsifiers, silicone emulsifiers, glyceryl monoesters, preferably glyceryl monoesters of C16-C22 saturated, unsaturated and branched chain fatty acids such as glyceryl oleate, glyceryl monostearate, glyceryl monopalmitate, glyceryl monobehenate, and mixtures thereof; polyglyceryl esters of C16-C22 saturated, unsaturated and branched chain fatty acids, such as polyglyceryl-4 isostearate, polyglyceryl-3 oleate, diglycerol monooleate, tetraglycerol monooleate and mixtures thereof; methyl glucose esters, preferably methyl glucose esters of C16-C22 saturated, unsaturated and branched chain fatty acids such as methyl glucose dioleate, methyl glucose sesquiisostearate, and mixtures thereof; sucrose fatty acid esters, preferably sucrose esters of C12-C22 saturated, unsaturated and branched chain fatty acids such as sucrose stearate, sucrose trilaurate, sucrose distearate (e.g., CRODESTA.RTM. F10), and mixtures thereof; C12-C22 ethoxylated fatty alcohols such as oleth-2, oleth-3, steareth-2, and mixtures thereof; hydrogenated castor oil ethoxylates such as PEG-7 hydrogenated castor oil; sorbitan ester ethoxylates such as PEG-40 sorbitan peroleate, Polysorbate-80, and mixtures thereof; polymeric emulsifiers such as ethoxylated dodecyl glycol copolymer; and silicone emulsifiers such as laurylmethicone copolyol, cetyldimethicone, dimethicone copolyol, and mixtures thereof.

Humectants:

Non-limiting examples of humectants include glycerin, propylene glycol, and laminaria digitata extract.

Further agents, which may be used, will be described in the following examples of various compositions and their manufacturing procedure in accordance with the teachings described herein. According to one exemplary manufacturing process, the first phase comprises the steps of adding citric acid to deionized water. The solution is then heated to approximately 80° C. Next, glycerin (99.5 natural USP), propylene glycol and Phenonip (phenoxyethanol, methylparaben, ethylparaben, propylparaben, butylparaben and isobutylparaben) are mixed into the solution to form a homogeneous mixture.

In the second phase of this exemplary process, Abil Wax 2434 (stearoxy dimethicone), steric acid and Generol 122N E-5 (PEG-5-Soy Sterol) are premixed and heated to a temperature of approximately 80° C. Once at 80° C., the phase 1 mixture is added to the mixture of phase 2 and mixed for approximately 15 minutes.

In phase 3 of this exemplary process, isosterarate isostearyle is mixed with Macadamia nut oil. Next, this premixture is heated to approximately 40° C. The cucumis melo fruit extract and hydrogenated vegetable oil is then added to the premixture. The mixture is mixed until all the solutes are dissolved and the mixture is uniform.

In phase 4 of this exemplary process, deionized water and Optigel CL (bentonite and cellulose gum) are mixed until uniform. At a temperature of approximately 40° C., the phase 4 mixture is added to the phase 3 mixture batch.

In phase 5 of this exemplary process, Kaolin USP 2747 is added to the phase 4 mixture. Subsequently, titanium dioxide 3328 is added to the mixture. In phase 6, the following components are individually added to the mixture: Laminaria Glycolic Ext “N”; Ginkgo Biloba extract (Ginkgo Biloba leaf extract and polypropylene glycol); Cucumber HS (propylene glycol and cucumis sativus fruit extract); sodium chondroitin sulfate; Eucalyptus Globulus leaf oil; Asparagopsis Armata extract; fragrance-parfum 8127 (available from Floressence); and Collagen Stimulation Factor MAP (water, denatured alcohol, lechithin, cholesterol, magnesium ascorbyl phosphate).

The various exemplary embodiments of the cosmetic compositions disclosed herein may also be made according to the following exemplary process. In phase 1, Veegum HS (magnesium aluminum silicate) is vigorously mixed with deionized water until the mixture is wetted and dispersed. During the mixing process, the mixture is also heated to a temperature of approximately 55° C. Next, Keltrol TF (Xanthan gum) is mixed with Glycerin 99.5 Natural USP 24 until the Xanthan gum is dispersed. During the heating of the Veegum mixture, the Keltrol mixture is added to the Veegum mixture. Next, Dipotassium Glycyrrhizinate is added to the mixture.

In phase 2 of this exemplary process, Lanol 1688 (Cetearyl Ethylhexanoate, available from Seppic) mixed with Liposorb S (Sorbitan Stearate), Liposorb S20 (Polysorbate 20), Cegermil (Cereal Germ Oil available from Libiol) and Vitamin E Acetate USP. During the mixing process, this mixture is heated to a temperature of approximately 55° C. The phase 2 premix is then added to the batch of phase 1 and mixed (for at least 10 minutes) until the mixture is uniform.

In phase 3 of this exemplary process, Sepigel 305 (polyacrylanide, C13-14 isoparaffin, Laureth-7) is mixed to the batch at a temperature of approximately 55° C. After the batch is uniformly mixed, the batch is then cooled.

In phase 4 of the exemplary process, Cegermil is mixed with Lanol 1688 and this premix is heated to a temperature of approximately 42° C. Once this mixture is uniform, the mixture is then cooled. Next, Ester OPC (palmitoyl grape seed extract, palmitic acid; available from Phytomedica) and Extramal (Cucumis Melo fruit extract, hydrogenated vegetable oil) are sieved and the larger particles are ground down as needed. With the phase 4 premix at approximately 33° C. and with vigorous mixing, the sieved Ester OPC and Extramel® are sprinkled into the phase 4 premix. The premix is then mixed until all components are dispersed. At approximately 33° C., the phase 4 premix is then added to the batch and mixed until uniform.

In phase 5 of the exemplary process, the following components are individually added to the batch mixture: Kigeline (water, butylene glycol, Kigelia Africana fruit extract, available from International Sourcing, Inc.); witch hazel water; Aloe Vera gel 10× (available from TCR Industries); and Bisabolol (available from TRI-K Industries).

In phase 6 of the exemplary process, Solubilisant Gamma 2420 (Octoxynol-11, polysorbate 20) is added to Parfum 8127 (available from Floressence) and Germazide MPB (phenoxyethanol, chlorphenesin, glycerin, Methylparaben, benzoic acid). Once the phase 6 premix has been uniformly mixed, the phase 6 premix is then added to the batch and subsequently mixed again.

In phase 7 of the exemplary process, collagen stimulation factor MAP (water, denatured alcohol, lecithin, cholesterol, magnesium ascorbyl phosphate) is added to the batch and mixed until uniform.

Yet another exemplary process may be used to form the exemplary embodiments of the cosmetic compositions disclosed herein. In phase 1, Veegum HS (magnesium aluminum silicate) is vigorously mixed with deionized water until the mixture is wetted and dispersed. During this mixing process, the mixture is also heated to a temperature of approximately 55° C. Next, Keltrol TF (Xanthan gum) is mixed with Glycerin 99.5 Natural USP 24 until the Xanthan gum is dispersed. Also, dipotassium glycyrrhizinate is mixed in deionized water until dissolved. During the heating of the Veegum mixture, the Keltrol mixture and the dipotassium glycyrrhizinate mixture are added to the Veegum mixture.

In phase 2 of this exemplary process, Lanol 1688 (Cetearyl Ethylhexanoate, available from Seppic) mixed with Liposorb S (Sorbitan Stearate), Liposorb S20 (Polysorbate 20), Cegermil (Cereal Germ Oil available from Libiol) and Vitamin E Acetate USP. During the mixing process, this mixture is heated to a temperature of approximately 55° C. The phase 2 premix is then added to the batch of phase 1 and mixed until uniform.

In phase 3 of this exemplary process, at a temperature of approximately 55° C., Sepigel 305 (polyacrylamide, C13-14 isoparaffin, Laureth-7) is added to the batch mixture. The batch mixture is mixed until a white viscous gel is formed.

In phase 4 of this exemplary process, Lanol 1688 is mixed with Dow Corning 200 Fluid, 100 cs (dimethicone), Ester DCM (dicaprylyl maleate, available from Bernel Co.), Vitamin E Acetate USP (tocopheryl acetate, available from Roche). This phase 4 premixture is then heated to 42° C., mixed until uniform and then cooled to approximately 32° C. Next, Extramel® (available from Seppic) is sieved, and then at a temperature of approximately 32° C. the sieved Extramel® is added to the phase 4 premix. The phase 4 premix is then added to the batch mixture and mixed until uniform.

In phase 5 of this exemplary process, hyaluronic acid FCH 1% Solution (water, sodium hyaluronate, phenoxyethanol) is added to deionized water. This phase 5 premixture is mixed until a clear gel is formed. This phase 5 mixture should take about 2 hours to make. Next, the phase 5 mixture is added to the batch mixture and mixed until uniform.

In phase 6 of the exemplary process, Solubilisant Gamma 2420 (Octoxynol-11, polysorbate 20) is added to Parfum 8127 (available from Floressence) and Germazide MPB (phenoxyethanol, chlorphenesin, glycerin, Methylparaben, benzoic acid). Once the phase 6 premix has been uniformly mixed, the phase 6 premix is then added to the batch and subsequently mixed again. In phase 7, the following components are individually added to the batch mixture: Phylliance (Phytic acid, available from Tri-K); Collagen Stimulation Factor MAP (water, denatured alcohol, lechithin, cholesterol, magnesium ascorbyl phosphate); and Pentacare-HP (water, Ceratonia siliqua gum, hydrolyzed casein, available from Centerchem).

Another exemplary process may be used to form the exemplary embodiments of the compositions disclosed herein. In phase 1, Carbopol 1342 (acrylates/C10-C30 alkyl acrylate crosspolymer) is mixed with deionized water. This mixture is then heated to 70° C and the following components are added to the phase 1 mixture: Disodium EDTA; Glycerin 99.5 Natural USP 24; and Botanistat PP (propylparaben, available from DD Chemco Inc.).

In phase 2 of this exemplary process, the following components are mixed together and heated to 70° C: Myritol 318 (caprylic triglyceride, available from Henkel Corporation); Gelot 64 (glceryl stearate, PEG-75 stearate, available from Gattefosse); Tefose 63 (PEG-6 stearate, glycol stearate, PEG-32 stearate, available from Gattefosse); Botanistate PP (propylparaben, available from DD Chemco Inc.); Parsol MCX (ethylhexyl methoxycinnamate, available from Roche Vitamins Inc.); and Parsol 1789 (butyl methoxydibenzoylmethane/avobenzone, available from Roche, Cosmetics & Specialty Chemicals Group). Once the phase 2 mixture is melted and uniform, the phase 2 mixture is added to the batch mixture from phase 1. In phase 3, at a temperature of approximately 70° C., sodium hydroxide pellets are added to the batch and mixed until uniform (for at least 15 minutes).

In phase 4 of this exemplary process, Parasol MCX and Myritol 318 are mixed together until uniform and heated to a temperature of approximately 42° C. The mixture is then cooled. Next, Extramel® is sieved and then added to the phase 4 mixture at a temperature of approximately 32° C. The resultant mixture is mixed until the Extramel® is dissolved. At 70° C., the phase 4 mixture is added to the batch and mixed (for at least 10 minutes) until uniform. The batch mixture is then cooled.

In phase 5 of this exemplary process, dipotassium glycyrrhizinate is dissolved in deionized water. At a temperature of approximately 40° C., the phase 5 solution is added to the batch mixture. In phase 6 of this exemplary process, the following components are individually added to the batch mixture: Acacia Collagen 97 (water, propylene glycol, Acacia Senegal extract, potassium sorbate, phenoxyethanol, methylparaben, ethylparaben, propylparaben, butylparaben, isobutylparaben, available from Scher); Ascorbosilane C (acorbyl methylsilanol pectinate, available from Exsymol); Herbasol Yarrow Extract (water, propylene glycol, Achillea Millefolium extract, sorbitol, phenoxyethanol, methylparaben, ethylparaben, propylparaben, butylparaben, isobutylparaben,available from Seltzer Chemicals); and Depigmentation Factor 2U (water, denatured alcohol, lecithin, cholesterol, arctostaphylos Uva Ursi leaf extract, available from Seltzer Chemicals). In phase 7, the following components are mixed together and then added to the batch mixture: Collagen Stimulation Factor MAP (water, denatured alcohol, lechithin, cholesterol, magnesium ascorbyl phosphate); Parfum 8127 (available from Florressence); and Germazide MPB R10355 (phenoxyethanol, chlorphenesin, glycerin, methylparaben, benzoic acid, available from Collaborative Laboratories).

In closing, it is to be understood that the exemplary embodiments described herein are illustrative of the principles of the present invention. Other modifications that may be employed are within the scope of the invention. Thus, by way of example, but not of limitation, alternative configurations may be utilized in accordance with the teachings herein. Accordingly, the drawings and description are illustrative and not meant to be a limitation thereof.

1. A skin care composition, comprising:
a safe and effective amount of a melon extract and magnesium ascorbyl phosphate (MAP), wherein the melon extract and the MAP minimize free radical damage to an individual's skin.

2. The skin care composition of claim 1 wherein the melon extract further comprises at least one antioxidant.

3. The skin care composition of claim 2 wherein the at least one antioxidant is selected from the group consisting of superoxide dismutase, catalase, vitamins, and mixtures thereof.

4. The skin care composition of claim 3 further comprising coenzyme Q1O, lipoic acid, glutathion, and mineral salts.

5. The skin care composition of claim 4 wherein the mineral salts is selected from the group consisting of potassium, magnesium, calcium, selenium, and mixtures thereof.

6. The skin care composition of claim 2 further comprising one or more skin care components, the skin care components being selected from the group consisting of conditioning agents, sunscreen actives, anti-acne actives, antimicrobial actives, antifungal actives, anti-wrinkle actives, anti-skin atrophy actives, skin repair actives, skin barrier repair actives, cosmetic soothing actives, artificial tanning actives, artificial tanning accelerators, skin lightening actives, sebum stimulators, sebum inhibitors, protease inhibitors, skin tightening agents, viscosity modifiers, emulsifiers, humectants, and mixtures thereof.

7. The skin care composition of claim 1 wherein the melon extract is a cucumis melo extract.

8. The skin care composition of claim 1 wherein the melon extract is coated into microgranules.

9. The skin care composition of claim 8 wherein the microgranules comprises approximately 50% melon extract and approximately 50% vegetable fat.

10. A skin care composition, comprising:
a safe and effective amount of a melon extract and magnesium ascorbyl phosphate (MAP), wherein the melon extract comprises at least one antioxidant, wherein the skin care composition for preventing and minimizing wrinkles.

11. The skin care composition of claim 10 wherein the at least one antioxidant is selected from the group consisting of superoxide dismutase, catalase, vitamins, and mixtures thereof.

12. The skin care composition of claim 11 further comprising coenzyme Q10, lipoic acid, glutathion, and mineral salts.

13. The skin care composition of claim 12 wherein the mineral salts is selected from the group consisting of potassium, magnesium, calcium, selenium, and mixtures thereof.

14. The skin care composition of claim 10 further comprising one or more skin care components, the skin care components being selected from the group consisting of conditioning agents, sunscreen actives, anti-acne actives, antimicrobial actives, antifungal actives, anti-wrinkle actives, anti-skin atrophy actives, skin repair actives, skin barrier repair actives, cosmetic soothing actives, artificial tanning actives, artificial tanning accelerators, skin lightening actives, sebum stimulators, sebum inhibitors, protease inhibitors, skin tightening agents, viscosity modifiers, emulsifiers, humectants, and mixtures thereof.

15. The skin care composition of claim 10 wherein the melon extract is a cucumis melo extract.

16. The skin care composition of claim 10 wherein the melon extract is coated into microgranules.

17. The skin care composition of claim 16 wherein the microgranules comprises approximately 50% melon extract and approximately 50% vegetable fat.

18. A skin care composition, comprising:
a safe and effective amount of a cucumis melo extract and magnesium ascorbyl phosphate, wherein the cucumis melo extract is formed into microgranules, and the cucumis melo extract includes at least one antioxidant.

19. The skin care composition of claim 18 wherein the at least one antioxidant is selected from the group consisting of superoxide dismutase, catalase, vitamins, and mixtures thereof.

20. The skin care composition of claim 19 further comprising coenzyme Q10, lipoic acid, glutathion, and mineral salts.

21. The skin care composition of claim 20 wherein the mineral salts is selected from the group consisting of potassium, magnesium, calcium, selenium, and mixtures thereof.

22. The skin care composition of claim 18 further comprising one or more skin care components, the skin care components being selected from the group consisting of conditioning agents, sunscreen actives, anti-acne actives, antimicrobial actives, antifungal actives, anti-wrinkle actives, anti-skin atrophy actives, skin repair actives, skin barrier repair actives, cosmetic soothing actives, artificial tanning actives, artificial tanning accelerators, skin lightening actives, sebum stimulators, sebum inhibitors, protease inhibitors, skin tightening agents, viscosity modifiers, emulsifiers, humectants, and mixtures thereof.

23. The skin care composition of claim 18 wherein the microgranules comprises approximately 50% melon extract and approximately 50% vegetable fat.

24. A method for treating an individual's skin, comprising:
providing a skin care composition as recited claim 1; and
topically applying the skin care composition to the individual's skin.
